(19) United States
(12) Patent Application Publication
(10) Pub. No.: US 2019/0024179 A1
(43) Pub. Date: Jan. 24, 2019

(54) METHOD FOR TESTING PERITONEAL DISSEMINATION OF GASTRIC CANCER BY EXPRESSION LEVEL OF SYT13, SYT8, OR ANOS1, TEST KIT, METHOD FOR SCREENING MOLECULARLY TARGETED THERAPEUTIC AGENT, AND THERAPEUTIC AGENT

(71) Applicant: National University Corporation Nagoya University, Aichi (JP)

(72) Inventor: Mitsuro KANDA, Aichi (JP)

(21) Appl. No.: 15/556,219

(22) PCT Filed: Mar. 4, 2016
(86) PCT No.: PCT/JP2016/056788

§ 371 Date: Sep. 6, 2017

(30) Foreign Application Priority Data
Mar. 6, 2015 (JP) 2015-044588
Aug. 13, 2015 (JP) 2015-159756

(51) Int. Cl. C12Q 1/6886; G16H 50/20; G16H 50/30

(52) U.S. Cl. CPC C12Q 1/6886; G16H 50/20; C12Q 2600/158; C12Q 2600/118; C12Q 2600/136; G16H 50/30

(57) ABSTRACT

Provided are: a method for predicting peritoneal dissemination of gastric cancer, and screening a drug that acts selectively on peritoneal dissemination; and a drug. SYT13, SYT8, and ANOS1, which are specifically expressed at high levels in groups with peritoneal dissemination of gastric cancer, are used as indicators, whereby it is possible to predict postoperative peritoneal dissemination and to screen a molecularly targeted therapeutic agent. Furthermore, these siRNAs can suppress peritoneal dissemination recurrence.

### BACKGROUND OF THE INVENTION

### 1. Field of the Invention

The present invention relates to a prognostic marker for gastric cancer, and a method for testing using the prognostic marker. The present invention more particularly relates to a method for testing gastric cancer that undergoes peritoneal dissemination, which affects the prognosis of gastric cancer, and relates to a test kit and a method for screening a molecularly targeted therapeutic agent. The present invention also relates to a drug for suppressing peritoneal dissemination.

### 2. Description of the Related Art

Gastric cancer occurs in significant numbers in Japan, China, South Korea, and other countries in Asia, and in South America. Looking at the site-specific cancer mortality in Japan, the mortality rate of gastric cancer has decreased annually, but is second only to lung cancer in men, and has the third-largest mortality rate following colon cancer, lung cancer in women (according to 2012 statistics). The mortality rate due to gastric cancer is decreasing due to early detection by the spread of cancer screening, yet advanced gastric cancer still has poor prognosis and is a significant disease to be overcome in Japan, which has a high incidence rate of gastric cancer.

Recurrent metastasis greatly affects the prognosis of gastric cancer. Chemotherapy is performed for recurrent gastric cancer, and for advanced gastric cancer for which recurrence is likely. The current standard regimen that is used is a treatment method based on administration of S-1 in which inhibition of DNA synthesis is the mechanism of action. As a result of a large cohort study, advanced gastric cancer is considered to be effectively treated by oral administration of S-1 as a postgastrectomy adjuvant therapy, and postoperative oral administration of S-1 is the standard treatment. S-1 is a reagent which is generally effective in cancers involving active in cell proliferation, and does not act specifically on gastric cancer. Although it has become possible to achieve a high tumor-reducing effect by chemotherapy, complete cure of recurrent gastric and advanced gastric cancer is difficult.

At present, gastric cancer having distant metastases are handled collectively, and there is no differentiation in treatment policy. However, there are three completely different routes to recurrent metastasis mode of gastric cancer, namely, peritoneal dissemination, hematogenous metastasis, and lymph node metastasis. A multistep process is required in order for the free cancer cells produced by the primary tumor to engraft and proliferate to metastatic focus, and adhesive molecules, proteolytic enzymes, growth factors, angiogenic factors, chemokines and many other molecules are reported to be involved. The three routes of recurrent metastases differ considerably in gastric cancer as well, and therefore, the molecules involved in metastasis are different, and the nature of the metastatic cancer cells is also thought to be very different. Nevertheless, the comparable treatment is carried out for remote metastasis which is established by three routes with different mechanisms, that is a cause of difficultly of a complete cure of metastatic cancer.

In recent years, identification of tumor markers having high specificity has been carried out by a comprehensive gene analysis method using a microarray and/or a next-generation sequencer. Methods for predicting and detecting recurrence and/or peritoneal dissemination in relation to gastric cancer have been disclosed (Patent Documents 1-3).

In addition, there has been increased vigor in the development of a molecularly targeted therapeutic agent capable of reducing side effects in the treatment of cancer. Molecularly targeted therapeutic agent refers to a reagent created so as to act efficiently using a protein or gene expressed on the surface as a target, the nature of cancer cells or other diseased cells having been ascertained at the molecular level. Molecularly targeted therapeutic agents for gastric cancer are few in number, and currently only Trastuzumab and Ramucirumab for HER2-positive gastric cancer have received approval in Japan.

### PRIOR ART DOCUMENTS

### Patent Documents

[Patent Document 1] Japanese Laid-open Patent Publication 2014-236726

[Patent Document 2] Japanese Laid-open Patent Publication 2004-321102

[Patent Document 2] Japanese Laid-open Patent Publication 2007-215412

### Non-Patent Documents

[Non-patent Document 1] Monterrat, C., et al., (2006), Biochim. Biophys. Acta, Vol. 1763, pp. 73-81
[Non-patent Document 2] Fukuda, M., & Mikoshiba, K., (2001), Biochem. J., Vol. 354, pp. 249-257
[Non-patent Document 3] Jian B., et al., (2009), Cell Cycle Vol. 8, pp. 3770-3776.
[Non-patent Document 4] Choy. C. T., et al., (2014), Endocr. Relat. Cancer, Vol. 21, pp. 85-99.

### SUMMARY OF THE INVENTION

As described above, although markers for evaluating the risk of recurrence and peritoneal dissemination have been disclosed, the precision is not very high and practical application has not yet occurred. Therefore, there is a need to identify a novel marker for evaluating recurrence risk with high precision. If a marker with high specificity can be found, more effective individualized treatments can be provided.

Trastuzumab, which has been approved for the first time in Japan as a molecularly targeted therapeutic agent for gastric cancer, is only effective for HER2-positive gastric cancer, which constitutes about 20% of advanced and recurrent gastric cancers. Also, Ramucirumab, which has been approved for treatment of non-excised, recurrent gastric cancer, is not considered to have high prognosis-extending effect. Consequently, there is a need to develop a novel molecularly targeted therapeutic agent for advanced gastric cancer that has remote metastasis or postresection recurrence risk.

An object of the present invention is to reveal genes involved in different recurrent metastatic forms of gastric cancer, to establish a novel marker capable of prognosis prediction, and to provide method and test kit for testing risk of peritoneal dissemination with a poor prognosis. Another object is to develop a method for screening a molecularly targeted drug using the marker as an indicator. A further object is to provide a therapeutic agent for suppressing peritoneal dissemination.

### Means for Solving the Problems

The present invention relates to the following testing method, kit, and screening method.

(1) A testing method for predicting postgastrectomy peritoneal dissemination, wherein a measurement is taken for an expression level of at least one of SYT13, SYT8, and ANOS1 in at least one sample of patient serum, peritoneal cavity lavage solution in a gastrectomy, and gastric tissue, collected from a subject, and risk of peritoneal dissemination is determined to be high when the expression level of at least one of SYT13, SYT8, and ANOS1 in the sample is higher than a predetermined value.

(2) The testing method of (1), wherein a method for measuring the expression level of SYT13, SYT8, and ANOS1 measures the expression level of SYT13, SYT8, and ANOS1 mRNA and/or protein.

(3) The testing method of (2), wherein the method for measuring the expression level of SYT13, SYT8, and ANOS1 mRNA is carried out by quantitative PCR.

(4) A test kit for diagnosing or predicting postgastrectomy peritoneal dissemination, wherein the kit comprises one or more of an anti-SYT13 antibody, an anti-SYT8 antibody, an anti-ANOS1 antibody, and a primer set for quantitative PCR for measuring an expression level of SYT13, SYT8, and ANOS1.

(5) A method for screening a molecularly targeted therapeutic agent by targeting SYT13, SYT8, ANOS1, wherein a substance is screened using expression, or suppression of function, of at least one of SYT13, SYT8, and ANOS1 as an indicator.

(6) A therapeutic agent for treating or preventing peritoneal dissemination of gastric cancer, wherein the therapeutic agent is obtained by the method for screening a molecularly targeted therapeutic agent set forth in (5).

(7) A drug composition for suppressing metastasis by postgastrectomy peritoneal dissemination, the drug composition comprising siRNA of at least one of SYT13, SYT8, and ANOS1.

### Effects of the Invention

In accordance with the present invention, the risk of occurrence of peritoneal dissemination can be predicted soon after gastrectomy, and this prediction can be used in treatment thereafter. Specifically, a follow-up plan can be formulated in view of postgastrectomy peritoneal dissemination and treatment can be carried out for a patient group with considerable risk of peritoneal dissemination. Furthermore, a drug can be screened using the expression level of at least any of SYT13, SYT8, and ANOS1 as an indicator. It is therefore possible to develop a drug for treating gastric cancer peritoneal dissemination in which these molecules are targets.

### BRIEF DESCRIPTION OF THE DRAWINGS

FIG. 1 is a diagram showing the results of a PCR array in which gastric cancer cells line has been used, FIGS. 1A and 1B show the results of a PCR array of genes with a correlation with SYT8 expression, and FIGS. 1C and 1D show the results of a PCR array of genes with a correlation with SYT13 expression;

FIG. 2 is a diagram showing the expression level of SYT8 and SYT13 of cells in a peritoneal cavity lavage solution, FIG. 2A shows the SYT8 expression level, and FIG. 2B shows the SYT13 expression level;

FIG. 3 shows the results of protein expression in gastric tissue by immunohistochemical staining, FIGS. 3A to 3C shows the result of protein expression of SYT8, and FIG. 3D to 3F shows that of SYT13;

FIG. 4 is a diagram comparing SYT13 mRNA expression level in tissue obtained from 200 cases of gastric cancer resection patients using the gastric cancer stage and the presence or absence of peritoneal dissemination;

FIG. 5 shows the correlation between SYT13 mRNA expression level in gastric cancer tissue and peritoneal dissemination, FIG. 5A shows the result of a correlation analysis using an ROC curve, and FIG. 5B is a comparison of later-occurring peritoneal recurrence frequency in 93 cases of Stage II/III gastric cancer in which radical gastrectomy had been performed;

FIG. 6 shows the measurement results of SYT13 mRNA expression level in a peritoneal fluid sample, FIG. 6A shows the result of a correlation analysis using an ROC curve, FIG. 6B shows the relationship between peritoneal dissemination recurrence and SYT13 expression, and FIG. 6C shows the relationship between SYT13 expression of the peritoneal fluid sample and the postoperative survival rate;

FIG. 7 is a diagram comparing SYT8 mRNA expression level in tissue obtained from 200 cases of gastric cancer resection patients using the gastric cancer stage and the presence or absence of peritoneal dissemination;

FIG. 8 shows the correlation between SYT8 mRNA expression level in gastric cancer tissue and peritoneal dissemination, FIG. 8A shows the result of a correlation analysis using an ROC curve, and FIG. 8B is a comparison of later-occurring peritoneal recurrence frequency in 93 cases of Stage II/III gastric cancer in which radical gastrectomy has been performed;

FIG. 9 shows the results of a knockdown experiment using siRNA, FIGS. 9A to 9C shows the results of suppressing SYT8 expression, FIGS. 9D to 9F show the results of suppressing SYT13 expression, and then an analysis of proliferation potency (FIGS. 9A, 9D), invasion potency (FIGS. 9B, 9E), and migratory potency (FIG. 9C, 9F);

FIG. 10 shows analysis results of weight loss after treatment involving SYT13 siRNA intraperitoneal administration in a murine peritoneal dissemination model;

FIG. 11 shows a comparison of laparotomy macroscopic findings 2 and 4 weeks after the start of treatment involving SYT13 siRNA intraperitoneal administration in a murine peritoneal dissemination model;

FIG. 12 shows a comparison of macroscopic findings of in vivo imaging 2, 4, and 6 weeks after the start of treatment involving SYT13 siRNA intraperitoneal administration in a murine peritoneal dissemination model;

FIG. 13 is a diagram in which the signal values of in vivo imaging of FIG. 12 have been quantified;

FIG. 14 is a diagram showing a survival curve of a murine peritoneal dissemination model in which treatment by SYT13 siRNA intraperitoneal administration has been carried out;

FIG. 15 is a diagram showing a survival curve of a murine peritoneal dissemination model in which treatment by SYT8 siRNA intraperitoneal administration has been carried out;

FIG. 16 shows the results of a PCR array using a gastric cancer cell line, and FIGS. 16A and 16B show genes which have a correlation with ANOS1 expression;

FIG. 17 shows the results of a knockdown experiment using ANOS1 siRNA, FIG. 17A shows the analysis results for proliferation potency, FIG. 17B shows that for invasion potency, and FIG. 17C shows that for migratory potency;

FIG. 18 shows the protein expression level in gastric tissue by ANOS1 immunohistochemical staining, FIG. 18A shows a typical immunohistochemical staining image, and FIG. 18B shows the correlation with the ANOS1 mRNA expression level;

FIG. 19 is a diagram showing the correlation between the ANOS1 expression level and the prognosis and stage, FIG. 19A shows the correlation of the ANOS1 mRNA expression level with the prognosis, FIG. 19B shows that with the stage, and FIG. 19C shows correlation of the ANOS1 protein expression level with the prognosis; and

FIG. 20 is a diagram showing the correlation of ANOS1 expression in serum with the stage and prognosis, FIG. 20A shows the correlation of ANOS1 protein quantity in serum with the stage, FIG. 20B shows an ROC curve analysis, and FIG. 20C shows the correlation with the prognosis.

### DESCRIPTION OF THE PREFERRED EMBODIMENTS

The present inventor classified postgastrectomy cases in accordance with follow-up data, and analyzed mRNA obtained from gastric cancer primary lesion tissue. As a result, it was found that SYT8, SYT13 is specifically and highly expressed in gastric cancer involved in peritoneal dissemination. Also, an expression analysis of mRNA and protein was carried out with respect to ANOS1, and a correlation with peritoneal dissemination was observed. Therefore, when the expression level thereof is at a certain level or higher, it is possible that peritoneal dissemination will occur.

SYT8 and SYT13 are proteins belonging to the Synaptotagmin (SYT) family. SYT family proteins are identified as calcium/phospholipid binding molecules present in synaptic vesicles, and have been suggested to function as calcium sensors. In humans, the presence of 17 isoforms has been reported, and these isoforms are reported to be distributed mainly in the brain tissue.

SYT8 is reported to be expressed in nerve cells, endocrine cells, and also in the pancreas and sperm (Non-patent Document 1). Also, SYT13, unlike other synaptotagmins, is reported to bind with phospholipids with or without calcium, and to be expressed in various tissues other than the brain (Non-patent Document 2).

However, there are currently no reports that SYT8 and SYT13 are highly expressed in gastric cancer peritoneal metastasis, which was discovered for the first time by the present inventor. So far, there has also been no report that not only SYT8 and SYT13, but also any of the synaptotagmin family molecules, are highly expressed in not only gastric cancer and gastric cancer peritoneal dissemination, but also in cancer cells.

ANOS1 (Anosmin-1) is an adhesive protein, which is a constituent component of the extracellular matrix (ECM). Primarily, expression in the brain and nervous system has been observed, and ANOS1 is known to promote the migration of gonadotrophin-releasing hormone (GnRH) neurons during generation (Non-patent Documents 3 and 4).

ECM and cell adhesion molecules are known to have a very important role in the growth of cancer cells, and in invasiveness such as epithelial-mesenchymal transition (EMT). However, there are many unknown factors regarding the role of ANOS1 in cancer. For example, expression of ANOS1 is known to be suppressed in cells of the lung and ovarian cancer (Non-patent Document 3). On the other hand, enhanced expression of ANOS1 is known to enhance integrin signals in brain tumors, thereby increasing malignancy, and is known to increase metastasis and resistance to apoptosis-inducing agents in colorectal cancer (Non-patent Document 3, 4). Thus, regarding the relationship between carcinogenesis and expression of ANOS1, various results have been obtained depending on the carcinoma, but no consistent conclusion has been reached. However, whatever the case may be, there are currently no reports related to the expression of ANOS1 in gastric cancer, and association with peritoneal metastasis in any carcinoma has not been known.

When the expression level of at least one of SYT13, SYT8, and ANOS1 in a sample of excised gastric cancer tissue or cells in a postgastrectomy peritoneal lavage solution is significantly higher than a predetermined value, it is predicted that there is a possibility that latent peritoneal dissemination is causing peritoneal dissemination even though visual confirmation cannot be made, or that the risk of future occurrence of peritoneal dissemination is high. Therefore, quantifying the expression level of at least one of SYT13, SYT8, and ANOS1 makes it possible to evaluate the risk of peritoneal dissemination.

Further, it is thought that SYT13/SYT8 is secreted from the primary tumor tissue of the stomach, or released into the blood from a destroyed tumor and circulated in the blood, and therefore it is possible to evaluate the risk of peritoneal dissemination by detection using ELISA or the like. Also, in relation to ANOS1, the present inventors found a correlation between detection of ANOS1 protein in serum and the stage and prognosis. Therefore, the risk of peritoneal dissemination can be evaluated by a blood test.

SYT13, SYT8, and ANOS1 can each be quantified for mRNA and/or protein, thereby making it possible to quantify the expression levels thereof. Measurement can be carried out by a quantitative PCR method for mRNA, and using an anti-SYT8 antibody, an anti-SYT13 antibody, and an anti-ANOS1 antibody for protein. Quantitative PCR methods that can be used are the SYBR Green method, the TaqMan probe method, the RT-PCR method, and other known methods. Also, protein can be quantified by the ELISA, the RIA, the Western blot, immunohistochemical staining, or other known methods. Measuring mRNA and protein makes it possible to quantify SYT13, SYT8, and ANOS1 expression levels with good sensitivity, and to evaluate the risk of peritoneal dissemination.

A correlation between the expression levels of SYT13, SYT8, and ANOS1 and the recurrence of gastric cancer peritoneal metastasis was observed, and therefore, the use of a kit containing a PCR primer set capable of detecting these genes, and/or an antibody capable of detecting proteins and a reagent required for detection, makes it possible to form a test kit for prognosis and prediction of recurrence of peritoneal metastasis after gastric cancer resection. This makes it possible to follow-up with great detail after surgery for advanced gastric cancer.

In addition to a PCR primer set capable of quantifying the expression levels of the genes, a kit for testing the expression levels of SYT13, SYT8, and ANOS1 may also include enzymes, reagents, and the like which are optimal for quantification. Any sequence can be used as long as the primer is capable of quantitatively measuring SYT13, SYT8, and ANOS1. Also, a control primer such as a primer that amplifies GAPDH may be included in order to normalize expression levels.

A kit for testing the expression levels of SYT13, SYT8, and ANOS1 proteins may include reagents required for detection, in addition to antibodies for detecting protein, an aptamer or the like, and molecules that bind to SYT13, SYT8, and ANOS1 proteins.

In patients of a gastric cancer peritoneal dissemination group, SYT13, SYT8, and ANOS1 are highly expressed, and therefore a molecularly targeted therapeutic agent can be selected by screening a compound that suppresses expression of these genes. Candidates for a molecularly targeted therapeutic agent can be selected from a library comprising low molecular weight compounds, natural products, and the like.

Furthermore, the siRNA used in the examples of the present invention suppresses the proliferation potency, invasion potency, and migratory potency of gastric cancer cell lines. Therefore, siRNA, which suppresses SYT13, SYT8, and ANOS1, can function as a therapeutic agent for suppressing peritoneal dissemination.

The present inventors performed intraperitoneal administration of SYT13, SYT8 siRNA in a murine peritoneal dissemination model, and found that peritoneal dissemination was suppressed. Therefore, it can be expected that peritoneal dissemination would be suppressed by administration of SYT13, SYT8 siRNA to a patient group with high risk of peritoneal dissemination.

SYT13, SYT8, and ANOS1 are membrane proteins or intercellular adhesion factors, and therefore, using an antibody against portions that are exposed extracellularly would mask the function thereof and peritoneal dissemination could be suppressed. Screening these antibodies would make it possible to create a new drug.

For example, a cell culture can be carried out by adding a library compound to a culture medium of a cell line in which SYT13, SYT8, and ANOS1 are highly expressed among gastric cancer cell lines and/or cancer cell lines established by gastric cancer peritoneal dissemination, and those compounds that suppress SYT13, SYT8, and ANOS1 expression can be selected. For example, cell lines with high expression of SYT13, SYT8, and ANOS1 include gastric cancer cell lines MKN1 and MKN45. The expression levels of SYT13, SYT8, and ANOS1 can be quantified by measuring mRNA and/or protein as described above, and therefore, a substance that suppresses expression with high sensitivity can be selected.

The following description is based on data below, and unless otherwise specifically stated, in the present invention, “SYT8 expression,” “SYT13 expression,” and “ANOS1 expression” refer to expression of genes and/or proteins.

### (1) Correlation Between SYT8, SYT13 and Peritoneal Dissemination

### Example 1

«Analysis of Genes that are Expressed Specifically in a Peritoneal Dissemination Recurrence Group»

Advanced gastric cancer cases in which a gastrectomy was performed at the Nagoya University School of Medicine were divided into four groups, namely, a five-years-or-more long-term recurrence-free group, a peritoneal dissemination recurrence group, a liver metastasis recurrence group, a lymph node recurrence group, and an analysis was carried out. First, in view of peritoneal dissemination recurrence, which accounts for about half the gastric cancer recurrence group and for which an effective treatment has not been established, detection of molecules having specific high expression in peritoneal dissemination recurrence cases was carried out. Specifically, therapeutic resection in stage III gastric cancer was carried out, and cases subjected to S-1 oral administration as postoperative adjuvant therapy were grouped in accordance with actual outcomes. The cases were divided into the four groups of the five-years-or-more long-term recurrence-free group, the peritoneal dissemination recurrence group, the liver metastasis recurrence group, the lymph node recurrence group, and expression profiling by transcriptome analysis was carried out using RNAs obtained from each four cases of gastric cancer primary tumor tissue.

RNA was extracted using an RNeasy kit (QIAGEN Co., Ltd.) from an obtained surgical sample. The extracted total RNA was prepared for a sequence library using a TruSeq RNA Sample Prep Kit (Illumina Co.) according to standard protocols.

Next, the Paired-End sequencing was performed using a next-generation sequencer Hiseq (Illumina Co., Ltd.) and transcriptome analysis was carried out. The read base length was 100 bases/read, the number of reference-acquired reads was 100 million read-pairs (200 million reads)/lane, and the reference acquisition data was acquired at a data acquisition rate of 20 Gb/lane.

Analysis was carried out by performing a mapping process to a specified reference sequence using HiSeq software, calculating the expression level of each gene based on FPKM (Fragments per kilobase of exon per million mapped sequence reads) values, and creating a comparison table between samples.

The expression level of 57,751 molecules were comprehensively analyzed by transcriptome analysis, and, in the peritoneal dissemination recurrence group, molecules which are highly expressed in comparison with the other three groups were detected. As a result, it was found that 22 genes are highly expressed. Selected from among these were analyzable genes that are not yet reported to have expression in cancer tissue and for which the function of the genes is reported. As a result, expression of SYT8 (NCBI RefSeq ID accession number: XM_005253216) and SYT13 (NCBI RefSeq ID accession number: NM_020826) was observed to increase predominantly in the peritoneal dissemination recurrence group, as shown in Table 1. Table 1 summarizes the calculated signal intensity ratio (log 2 ratio) of each group in which metastasis was observed against the long-term recurrence-free group.

TABLE 1

| | Group | | | | | |
|---|---|---|---|---|---|---|
| | Peritoneal dissemination | | Liver metastasis | | Lymph node | |
| | recurrence-free group | | group | | recurrence group | |
| Gene | log2 | p value | log2 | p value | log2 | p value |
| SYT8 | 3.25484 | 0.0001 | −0.422228 | 0.5565 | −0.463201 | 0.5075 |
| SYT13 | 2.48272 | 0.00005 | −0.097281 | 0.8364 | 0.0729381 | 0.88085 |

As shown in Table 1, SYT8 and SYT13 are both observed to have significantly enhanced gene expression in the peritoneal dissemination recurrence group relative to the long-term recurrence-free group. In cases where peritoneal dissemination recurred, SYT8 was observed to have about 8-fold enhanced expression, and SYT13 was about 5.6-fold enhance expression in comparison with the recurrence-free group. In contrast, enhanced expression of SYT8 and SYT13 was not observed in the liver metastasis recurrence group and the lymph node recurrence group, which are other forms of metastasis. Therefore, SYT8 and SYT13 gene expression levels and protein expression levels function as biomarkers for gastric cancer peritoneal dissemination.

### Example 2

«Expression Analysis in Cell Lines»

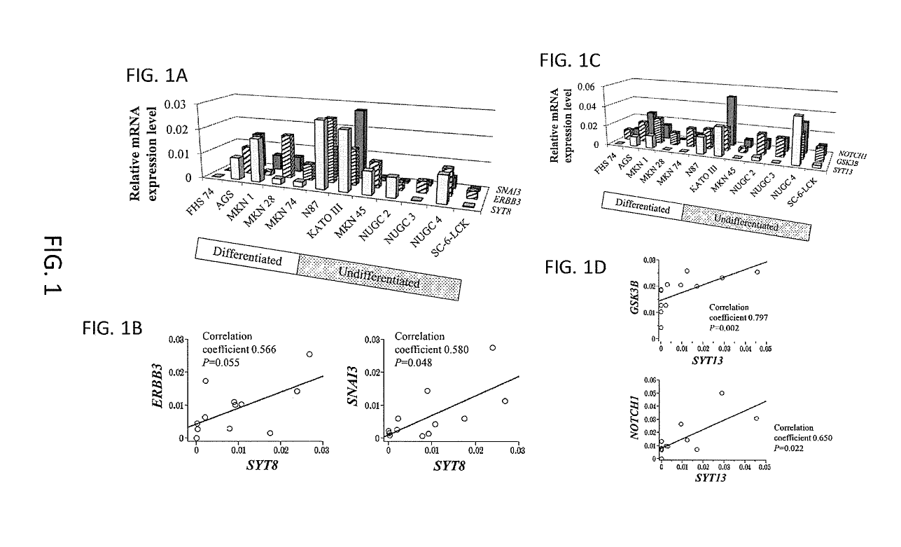

A PCR array analysis was performed on 11 types of gastric cancer cell lines and non-cancer epithelial cell line FHs 74. Expression of 84 genes (EMT, transcription factors, extracellular matrix, adhesion factor, genes involved in cancer-related major routes) was comprehensively analyzed using twelve cell lines by Human Epithelial to Mesenchymal Transition (EMT) RT² Profiler PCR Array (Qiagen Inc.). A correlation test was carried out between the results and the expression level of the SYT8 and SYT13 in each cell line. FIGS. 1A and 1B show the results for SYT8, and FIGS. 1C and 1D show the results for SYT13.

Expression of SYT8 had a significant positive correlation with ERBB3 (HER3), which is a tyrosine kinase known to be involved in the proliferation of cancer cells, and EMT-related transcription factor SNAI3, which is important in cancer cell metastasis and invasion. This result suggests that SYT8 is cooperatively expressed with known major cancer-related molecules and possibly promotes gastric cancer peritoneal dissemination from interrelations therewith.

Expression of SYT13 had a significant positive correlation with signal transduction molecule GSK3B, which has an important role in cell proliferation, and NOTCH1, which is an important EMT-related transcription factor in cancer cell metastasis and invasion. This result suggests that SYT13 is cooperatively expressed with known major cancer-related molecules and possibly promotes gastric cancer peritoneal dissemination from interrelations therewith.

### Example 3

«Correlation of Latent Peritoneal Dissemination and Expression of SYT8 and SYT13 in Gastric Cancer Tissue»

A correlation analysis between SYT8 and SYT13 expression and latent peritoneal dissemination was carried out in cases in which peritoneal dissemination was not visually observed during surgery.

Cells in a peritoneal cavity lavage solution were subjected to Papanicolaou staining and Giemsa staining, and a diagnosis made for positive and negative peritoneal dissemination. Quantitative PCR was performed using the following primer sequences and an analysis was carried out by amplification in PCR conditions of 40 cycles at 95° C. for 5 seconds and 60° C. for 60 seconds following heating at 95° C. for 10 minutes using an ABI STEPOnePlus Real-Time PCR System (Applied Biosystems, Inc.).

Also, GAPDH was used as a control to normalize each RNA value. The quantitative PCR primers for GAPDH that were used are noted below. The PCR conditions used for amplification were heating at 95° C. for 10 minutes, then 40 cycles at 95° C. for 5 seconds and 60° C. for 60 seconds.

Primer Sequences

SYT8:
(SEQ ID NO: 1)
Forward GCTTCTCTCTCCGGTACGTG
(SEQ ID NO: 2)
Reverse AGGAAGGTGAAGGCCTCATT
SYT13:
(SEQ ID NO: 3)
Forward ACCTGGAGAAGGCGAAGC
(SEQ ID NO: 4)
Reverse TCTGGGAACTTGAGGAGGG
GAPDH:
(SEQ ID NO: 5)
Forward GAAGGTGAAGGTCGGAGTC
(SEQ ID NO: 6)
Reverse GAAGATGGTGATGGGATTTC

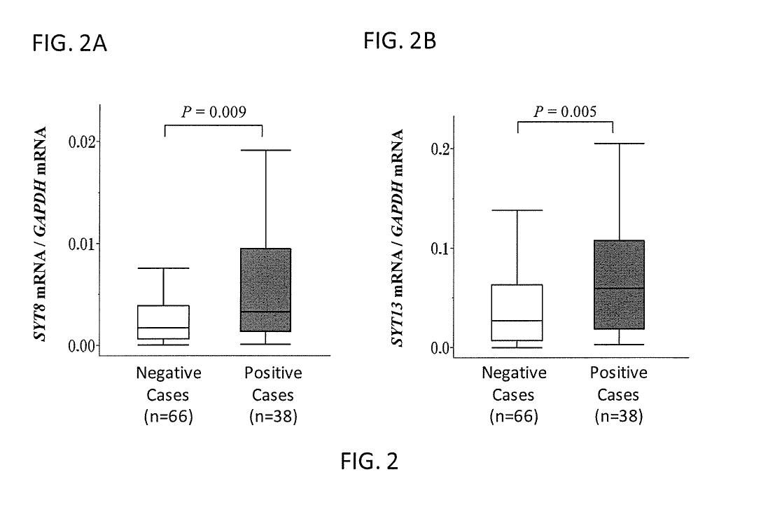

A pathological diagnosis of peritoneal dissemination lavage solution collected during surgery was carried out for 104 cases of gastrectomy. The cases were divided into 66 cases that were determined to be negative in a cyto-diagnosis of a peritoneal dissemination lavage solution, and 38 cases that were determined to be positive for latent peritoneal dissemination. The expression of SYT8 and SYT13 in gastric cancer tissue was analyzed using quantitative PCR. The results are shown in FIG. 2.

FIG. 2A shows values obtained by normalizing the expression level of SYT8 mRNA with the expression level of GAPDH mRNA in a box-and-whisker plot (minimal value, first quarter point, median value, third quarter point, and maximum value) of each group of cases with a negative cyto-diagnosis and cases with a positive cyto-diagnosis. FIG. 2B similarly shows the expression level of SYT13. Although metastasis was not visually observed, the expression levels of SYT8 and SYT13 were significantly high in cases determined to be cancer cell positive in a peritoneal cavity lavage solution and in which peritoneal dissemination was observed.

Although there are variations by patient, a comparison of the positive and negative cyto-diagnosis groups showed that the expression levels SYT8 and SYT13 were both significantly high in the positive group. Although optimal cutoff values are preferably verified using many samples, risk prediction of gastric cancer in which peritoneal dissemination recurs is made possible by measuring the SYT8 and SYT13 expression levels. For example, SYT8 and SYT13 are both about 25% for negative patients in excess of the median value of the positive cyto-diagnosis group, as shown in FIG. 2, and therefore the risk of gastric cancer in which peritoneal dissemination recurs can be predicted with reference to the median values of the positive cases. Cutoff values are expected differ depending on the method for measuring SYT13 and SYT8 and on the samples to be measured. Therefore, it is preferred that appropriate cutoff values are obtained, patients with a high risk of peritoneal dissemination recurrence are selected, and follow-up is carried out.

### Example 4

«Analysis of SYT8 and SYT13 Protein Expression in Gastric Tissue by Immunohistochemistry Staining»

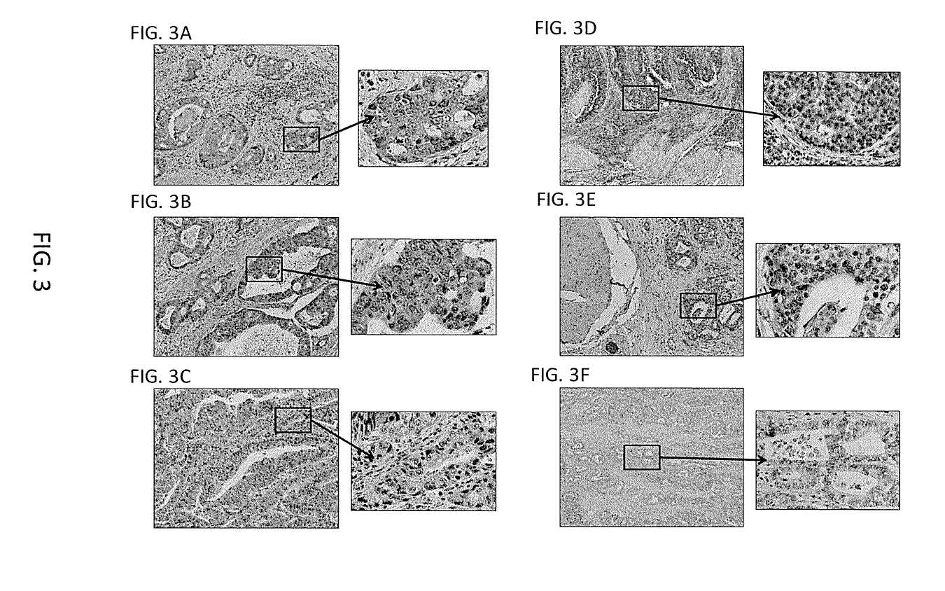

SYT8 and SYT13 were both studied using the 60 cases of patient tissue. Staining was carried out using an anti-SYT8 antibody (LifeSpan BioSciences Corp.), an anti-SYT13 antibody (Aviva Systems Biology Corp.), a Histofine SAB-PO kit (Nichirei Corp.) as a biotinylated secondary antibody kit, and a DAB substrate kit (Nichirei Corp.). FIG. 3 shows images obtained by staining by antibodies, and thereafter staining by hematoxylin.

In cases with high expression of SYT8 in gastric cancer tissue, significantly high peritoneal dissemination was observed in comparison with cases with low expression of SYT8 (53% vs. 4%, P<0.001). FIGS. 3A to 3C show typical staining images. FIGS. 3A and 3B show cases of positive peritoneal dissemination, which is positive expression of the SYT8 protein, and FIG. 3C shows a case of negative peritoneal dissemination, which is negative expression of the SYT8 protein.

In cases with high expression of SYT13 in gastric cancer tissue, significantly high peritoneal dissemination was observed in comparison with cases with low expression of SYT13 (28% vs. 0%, P<0.001). FIGS. 3D to 3F show typical staining images. FIGS. 3D and 3E show cases of positive peritoneal dissemination, which is positive expression of the SYT13 protein, and FIG. 3F shows a case of negative peritoneal dissemination, which is negative expression of the SYT13 protein.

It is apparent from these results that the extent of SYT8 and SYT13 protein expression in gastric cancer tissue is useful in prediction and diagnosis of peritoneal dissemination.

Cyto-diagnosis of gastric cancer tissue and together with confirming expression of SYT8 and SYT 13 by immunostaining makes it possible to perform prognosis and prediction with greater precision. Next, it was studied whether peritoneal dissemination can be predicted by SYT8 and SYT 13 expression in gastric cancer tissue at the time of resection using a patient sample. First, result of analysis of SYT13 expression will be shown.

### Example 5

«Comparison of SYT13 mRNA Expression Levels in Tissue Obtained from Gastric Cancer Resection Patients»

SYT13 mRNA was measured by quantitative PCR and expression levels were compared for mRNA in tissue obtained from 200 cases of gastric cancer resection patients.

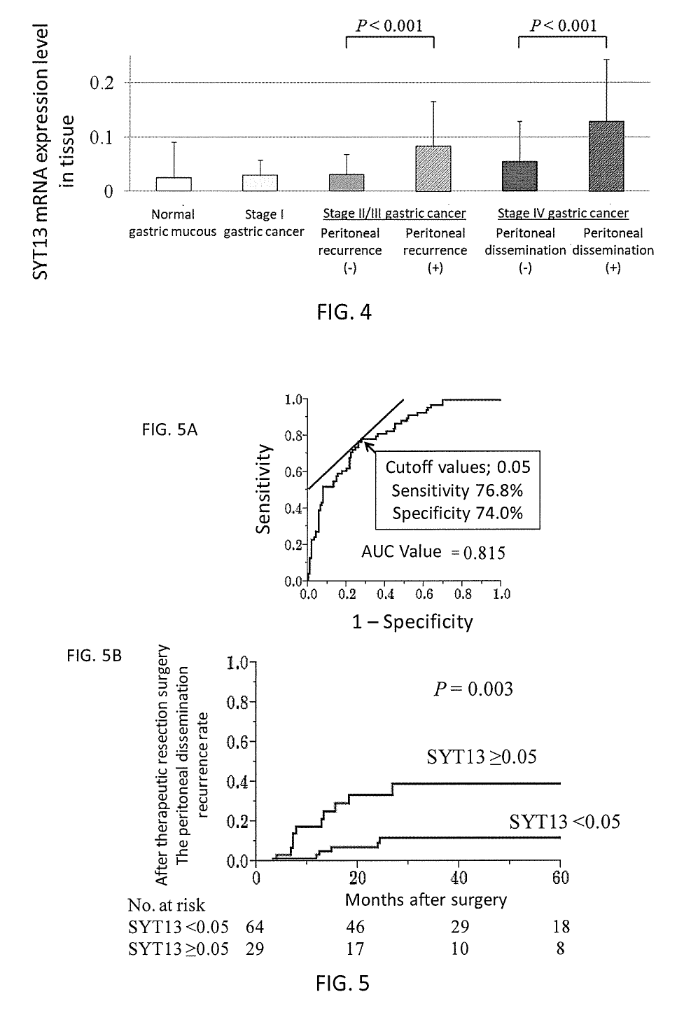

The patient groups were classified by stage, from stage I to stage IV, patients in stage II to stage IV were further finely classified by whether cases later involved recurrence of peritoneal dissemination, and the SYT13 mRNA expression levels were compared. The results are shown in FIG. 4. The expression level of SYT13 was a low value in early stage I gastric cancer tissue similar to normal gastric mucous membrane tissue. In stage II/III gastric cancer which has performed therapeutic resection, the SYT13 expression level in gastric cancer tissue at the time of surgery for cases that later involved recurrence of peritoneal dissemination was significantly higher than cases that peritoneal dissemination does not recur. In stage IV cases in which liver metastasis, peritoneal dissemination, remote lymph node metastasis and other remote metastases had already occurred at the time of surgery, the expression level of SYT13 in gastric cancer tissue was significantly high the cases with peritoneal dissemination in comparison with cases with other metastases. In view of the above, it was shown that measuring the expression level of SYT13 in gastric cancer tissue makes it possible to evaluate the risk of future peritoneal dissemination recurrence in addition to evaluation of the presence of peritoneal dissemination at that point in time.

The correlation between peritoneal dissemination and the expression level of SYT13 mRNA in the gastric cancer tissue obtained from above 200 cases of gastric cancer resection patients was analyzed for degree of correlation using an ROC curve. The results are shown in FIG. 5A. The value of the area under the curve was 0.815 and a very strong correlation was shown between peritoneal dissemination and the expression level of SYT13 mRNA in the gastric cancer tissue. The optimum cutoff value of the expression levels of SYT13 was calculated to be 0.05.

Next, patients were divided into two groups by the cutoff value of SYT13 expression obtained in FIG. 5A, and the future peritoneal dissemination recurrence frequencies were compared for 93 cases of stage II/III gastric cancer that underwent radical gastrectomy. Peritoneal dissemination was not detected at the time of gastric cancer resection in the 93 cases of stage II/III gastric cancer. The peritoneal dissemination recurrence rate for the patients divided into two groups by the cutoff value was plotted in relation to the number of months after surgery (FIG. 5B). In the case group having a SYT13 expression level in gastric cancer tissue of 0.05 (cutoff value) or higher, peritoneal dissemination recurred earlier and at a higher frequency. This shows that the SYT13 expression level is a useful biomarker in terms of both present diagnosis and prediction of peritoneal dissemination.

### Example 6

«SYT13 mRNA Expression Levels in Peritoneal Fluid Sample»

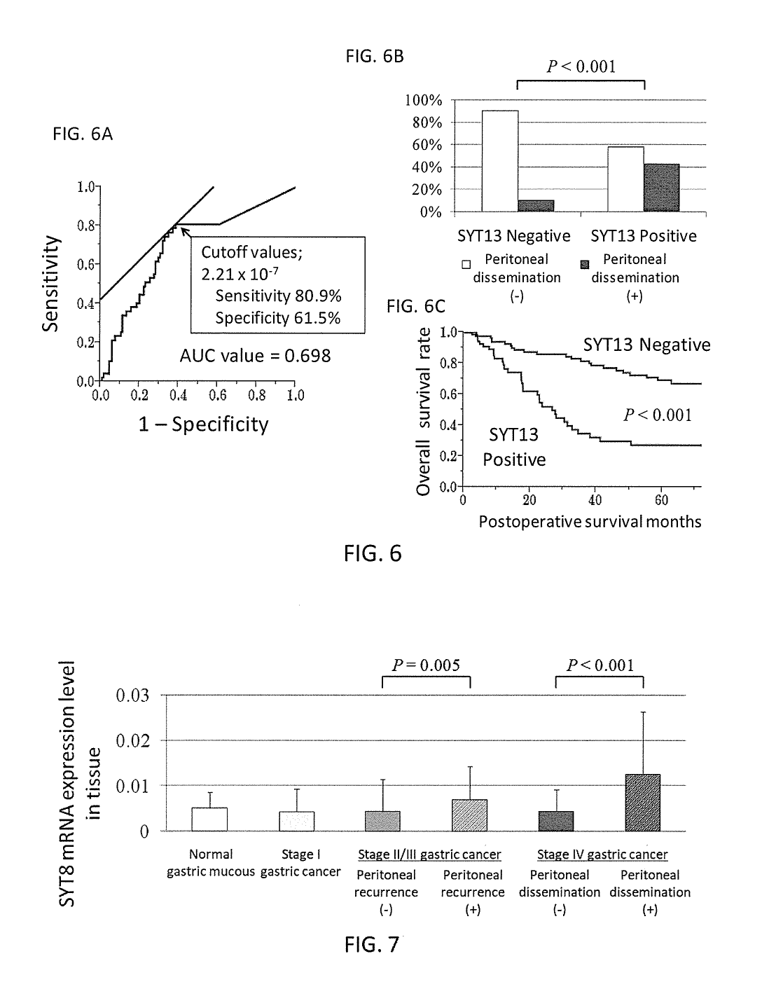

The SYT13 mRNA level in peritoneal fluid samples obtained from a patient group of 182 cases, which are different from the 200 cases of gastric cancer patients described above, was measured by quantitative PCR. The SYT13 mRNA expression level was measured using the mRNA obtained from cells in peritoneal fluid, and the correlation with peritoneal dissemination was analyzed using an ROC curve (FIG. 6A). The value of the area under the curve was 0.698 and peritoneal dissemination recurrence was shown to have a strong correlation with SYT13 mRNA expression. The optimum cutoff value of the expression levels of SYT13 was calculated to be 2.21×10⁻⁷. Also, the frequency of cases of peritoneal dissemination positive was high in the cases of SYT13 positive in peritoneal fluid, as shown in FIG. 6B.

The patients were divided into two groups by the cutoff value obtained in FIG. 6A, and the survival rate of all the 182 gastric cancer cases for which the peritoneal fluid samples were analyzed above were compared. The group that was positive for SYT13 in peritoneal fluid (expression level: 2.21×10⁻⁷ or higher) had significant poor prognosis. Therefore, SYT13 was shown to be a promising biomarker in not only gastric tissues, but also in expression level in patient peritoneal fluid.

### Example 7

«Comparison of SYT8 mRNA Expression Levels in Tissues Obtained from the Gastric Cancer Resection Patients»

Next, the results of analyzing SYT8 mRNA in tissue obtained from gastric cancer resection patients are shown. SYT8 mRNA was measured by quantitative PCR and expression levels were compared in the same manner as Example 5 for mRNA in tissue obtained from 200 cases of gastric cancer resection patients (FIG. 7).

From stage I which is early stage, to stage IV which is accompanied remote metastasis, the expression level of SYT8 in gastric cancer tissue in cases unaccompanied with peritoneal dissemination, was at the same level as normal gastric mucous membrane tissue. In stage II/III gastric cancer, which have performed therapeutic resection, the expression level of SYT 8 in gastric cancer tissue at the time of surgery for cases involving peritoneal dissemination recurrence was a significantly higher value than when there was no peritoneal dissemination recurrence. In stage IV cases, which already had liver metastasis, peritoneal dissemination, remote lymph node metastasis, or other remote metastasis at the time of surgery, the expression level of SYT8 in gastric cancer tissue was a significantly higher value in cases having peritoneal dissemination than cases having other metastases. It was therefore shown that measuring the expression level of SYT8 in gastric cancer tissue in the same manner as SYT13 makes it possible to evaluate the future risk of peritoneal dissemination in addition to the presence of peritoneal dissemination at that point in time.

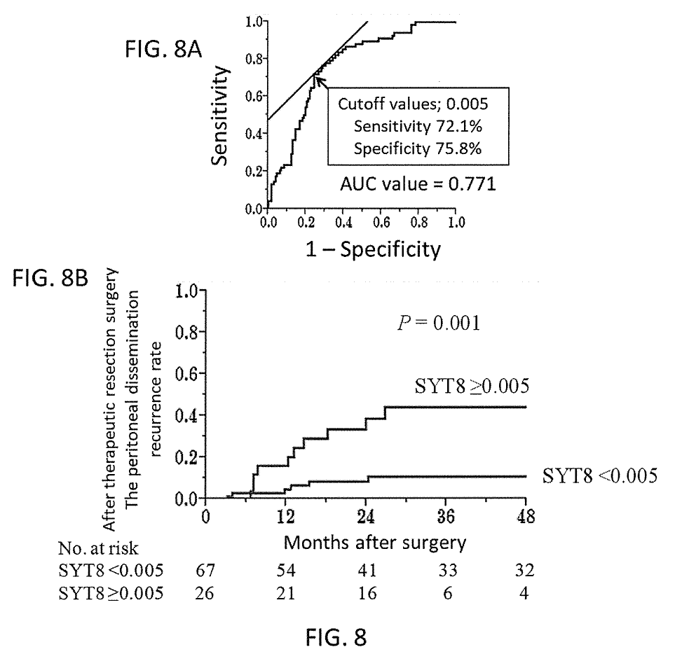

The correlation between peritoneal dissemination and the SYT8 mRNA expression level in gastric cancer tissue obtained from the above 200 cases of gastric cancer resection patients was analyzed using an ROC curve. The results are shown in FIG. 8A. The value of the area under the curve was 0.771 and a very strong correlation was shown between peritoneal dissemination and the expression level of SYT8 mRNA in the gastric cancer tissue. The optimum cutoff value of the expression levels of SYT8 was calculated to be 0.005 (FIG. 8A).

Next, patients were divided into two groups by the cutoff value obtained above, and the future peritoneal dissemination recurrence frequencies were compared for 93 cases of stage II/III gastric cancer that underwent radical gastrectomy. Peritoneal dissemination was not detected at the time of gastric cancer resection in the 93 cases of stage II/III gastric cancer. The peritoneal dissemination recurrence rate for the patients divided into two groups by the cutoff value was plotted in relation to the number of months after surgery (FIG. 8B). In the case group having a SYT8 expression level in gastric cancer tissue of 0.005 or higher, peritoneal dissemination recurred earlier and at a higher frequency. This shows that the SYT8 expression level is a useful biomarker in terms of both present diagnosis and prediction of peritoneal dissemination.

### Example 8

«Knockdown Analysis Using siRNA»

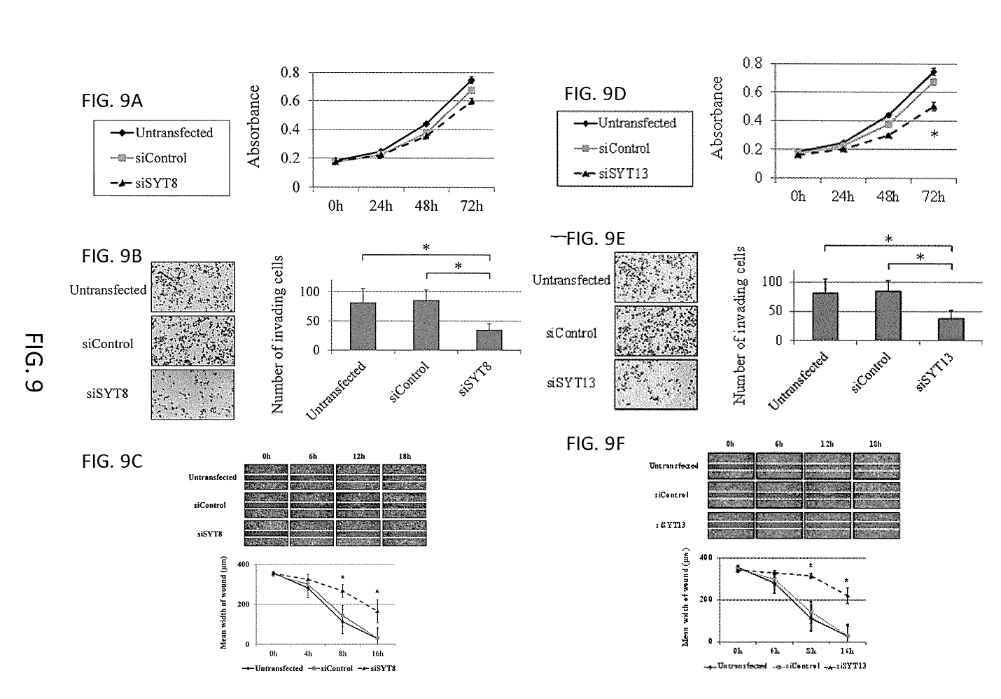

A strong correlation was observed between peritoneal dissemination and the expression of SYT8 and SYT13, and therefore, an experiment to selectively inhibit of expression (knockdown) of the genes was carried out using gastric cancer cell lines (MKN1, MKN45), which have high expression of SYT8 and SYT 13 in vitro, and the proliferation potency, invasion potency, and migratory potency of gastric cancer cells were evaluated. FIGS. 9A to 9C show the results using SYT8 siRNA, and FIG. 9D to 9F show the results using SYT13 siRNA. FIG. 9 shows the results obtained using the MKN1, and similar results were obtained using MKN45.

Using Accell siRNA transfection methods (Dharmacon, Inc.), siRNA was transfected to MKN1 and MKN45 cells, the cells were incubated for 72 hours in a serum-free DMEM medium, proliferation potency, invasion potency, and migratory potency were evaluated. SYT8 siRNA, SYT13 siRNA, and control siRNA (Accell Green Non-targeting) were each obtained from Dharmacon, Inc.

The cells cultured for 72 hours in the serum-free medium after siRNA transfection were evaluated in the following manner for cell proliferation potency. 1×10⁴ cells of MKN1 and MKN45 were disseminated in 96-well plates, respectively, and cultivated for 96 hours in a DMEM medium to which 2% fetal bovine serum had been added, 10 μL of Premix WST-1 Cell Proliferation Assay System (Takara Bio Inc.) was added, and absorbance was measured 24 hours later.

The results are shown in FIG. 9A (SYT8) and FIG. 9D (SYT13). It was apparent that when expression of both SYT8 and SYT 13 is inhibited, proliferation potency of gastric cancer cells is mildly suppressed.

The invasion potency of cells was evaluated by a Matrigel invasion assay of the cells cultured for 72 hours in a serum-free medium after siRNA transfection. An assay was carried out in accordance with protocol using BioCoat Matrigel Invasion Chambers (BD Siosciences Co.). Specifically, MKN1 and MKN45 cells were disseminated 2.5×10⁴ per well and incubated for 24 hours in a serum-free DMEM medium, the cells at the membrane bottom were then immobilized, staining was carried out using Diff-Quick (Sysmex Corp.), and the cells were observed and counted under a microscope. Microscopic observation was carried out at a magnification of 200× to obtain an average and standard deviation of five randomly selected fields of view.

A microscope image is shown on the left in FIG. 9B (SYT8) and FIG. 9E (SYT13), and the number of cells having invasion potency is shown on the right. It was apparent that when expression of both SYT8 and SYT 13 is inhibited, there is a significant suppression of invasion potency of gastric cancer cells.

Migratory potency of cells was evaluated by a wound-healing assay using cells cultured for 72 hours in a serum-free medium after siRNA transfection. 2×10⁴ cells of MKN1 and MKN45 respectively were disseminated in 12-well plates which are formed a wound gap to a predetermined width with the ibidi Culture-insert method (ibidi Inc.), and were cultivated in a serum-free medium. The insert was removed after 24 hours of dissemination and the wound width was measured in 200 μm intervals every six hours. Measurement was carried out using a microscope at a magnification of 40× and for each well in 10 locations to determine the average and standard deviation.

FIGS. 9C (SYT8) and 9F (SYT13) show the change over time of the microscope image and the wound width. It is apparent that when expression of both SYT8 and SYT13 is inhibited, there is a significant suppression of invasion potency of gastric cancer cells. These results suggest the possibility that SYT8 and SYT13 are involved in migratory potency and invasion potency of gastric cancer cells, and that inhibiting SYT8 and/or SYT 13 makes it possible to suppress metastasis of gastric cancer cells.

### Example 9

«Analysis Using Murine Peritoneal Dissemination Model»

From the results using cultured cells, it is suggested that SYT8 and SYT13 expression may be a cause of peritoneal dissemination. Therefore, an analysis was carried out using a murine model to determine whether peritoneal dissemination recurrence is suppressed by inhibition of SYT8 and SYT13 expression using siRNA.

To obtain a peritoneal dissemination model, 1×10⁶ human gastric cancer cells, MKN45 cells, which transfected with a luciferase gene, were intraperitoneally injected to immunodeficient mice (BALBc nu/nu, male, 10 weeks old). After the MKN45 cells were intraperitoneally injected, a 50 μg/5 μL siRNA solution, a 80 μL in vivo transfection reagent for experiment use (LEO-10, Hokkaido System Science Co., Ltd.), and 415 μL of a 5%-glucose solution for a total of 500 μL was administered twice a week over six weeks. The siRNA used was manufactured by Dharmacon Inc., the same as Example 8. The control group was administered 500 μL of a 5%-glucose solution. The experiment was conducted using nine mice each in the control group and the siRNA intraperitoneal administration group.

Luciferin (150 mg/kg) was intraperitoneally administered at 2, 4, and 6 weeks after cell injection, the amount of luminance was measured 15 minutes thereafter using In Vivo Imaging System (IVIS) Lumina (Xenogen Corp.), and the proliferation of cancer cells was analyzed.

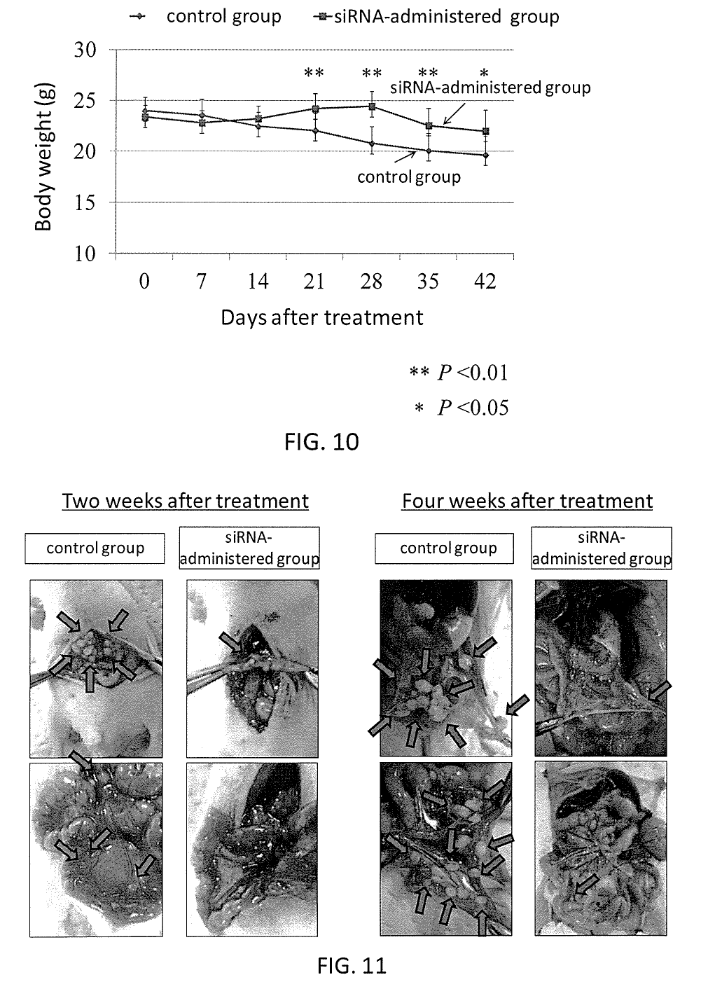

First, the effect on body weight of model animal administered with SYT13 siRNA will be shown. FIG. 10 shows the changes in body weight between the control group and the SYT13 siRNA administration group after intraperitoneal introduction of cancer cells. Weight loss over time due to progression of cancer in the control group was observed. On the other hand, body weight was significantly maintained in SYT13 siRNA administration group. This indicates that, in addition to disease activity of peritoneal dissemination being suppressed, siRNA administration does not have a harmful side effect on mice.

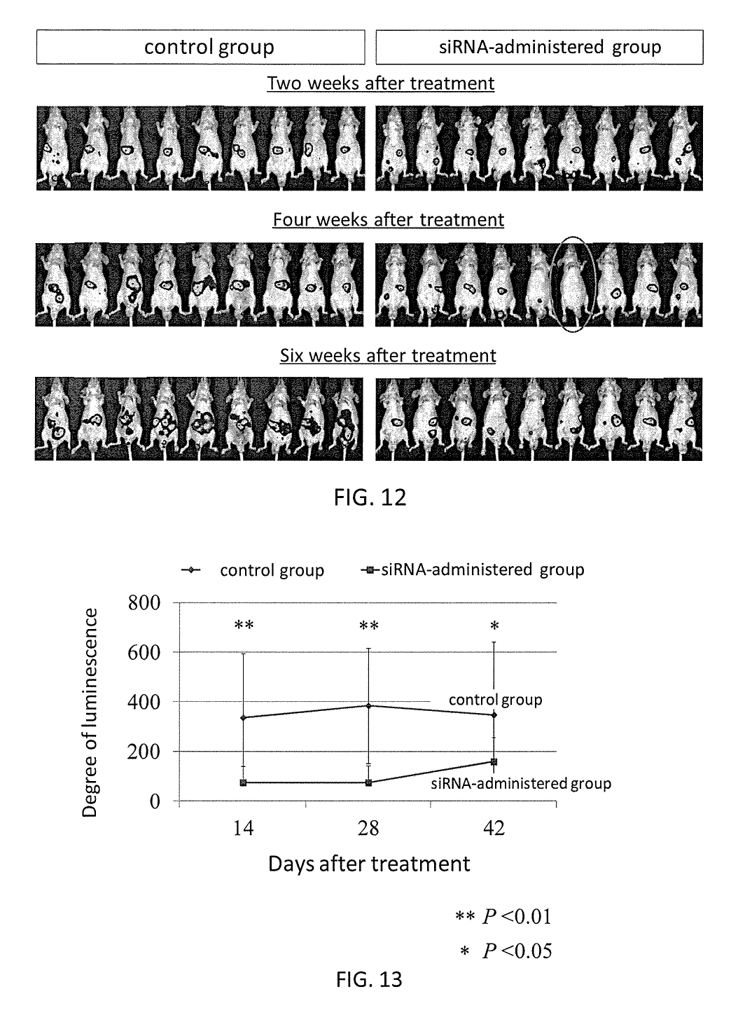

FIG. 11 shows the macroscopic findings of laparotomy 2 and 4 weeks after the start of treatment. The white peritoneal nodules indicated by arrows are markedly observed in the control group, whereas in siRNA-administered group, there was a clear reduction. FIG. 12 shows the findings of in vivo imaging 2, 4, and 6 weeks after the start of treatment. The photographs of mice shown in FIG. 12 are arranged in sequence from first to ninth of each group in each week. Therefore, mice lined up in each group vertically are the same individual. In the SYT13 siRNA-administered group, an increase over time in the signal is suppressed in comparison with the control group. Notably, mice for which the signal once disappeared 4 weeks after treatment of siRNA administration were also confirmed (circled). FIG. 13 is a comparison obtained by quantification of the signal value of the in vivo imaging of FIG. 12. In relation to the degree of luminescence, the siRNA-administered group had a significant low value in comparison with the control group at all time points of 2, 4, and 6 weeks after treatment.

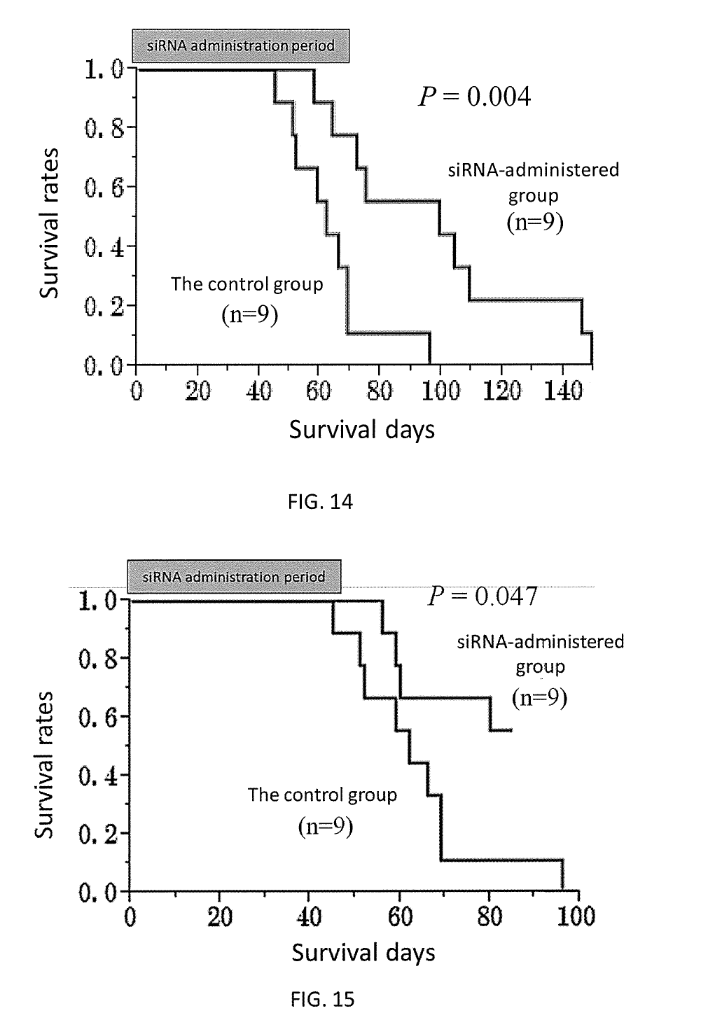

FIG. 14 shows the survival curve of the above-described mice. In comparison with the control group, the survival period for mice was significantly extended by SYT13 siRNA administration. Therefore, SYT13 can be used not only in diagnosis of prognosis, but can also suppress peritoneal dissemination recurrence using a reagent that reduces the expression thereof in the manner of siRNA.

### Example 10

«Analysis Using Murine Peritoneal Dissemination Model»

The therapeutic effect of SYT8 siRNA intraperitoneal administration was examined in the same manner as Example 9. SYT8 siRNA was administered twice a week over six weeks in murine peritoneal dissemination model in the same manner as described above. The siRNA used was manufactured by Dharmacon Inc., the same as Example 8. The experiment was conducted using nine mice each in the control group and the siRNA intraperitoneal administration group.

FIG. 15 shows the survival curve. The survival period was significantly extended by SYT8 siRNA administration. Five mice in nine still survived even after 102 days in SYT8 siRNA-administered group, whereas all the mice in the control group died on the 95th day.

### (2) Correlation Between ANOS1 and Peritoneal Dissemination

### Example 11

«Presence or Absence of Peritoneal Dissemination and Expression of ANOS1»

Next, the analysis results for ANOS1 are shown. The relationship between ANOS1 (NCBI RefSeq ID accession number: XM_006190153.1) mRNA expression and peritoneal dissemination was analyzed. As described above, the results suggest that ANOS1 expression is related to cancer cell proliferation and epithelial-mesenchymal transition (EMT), but there is also contradictory data. Therefore, the correlation between ANOS1 and peritoneal dissemination in gastric cancer was analyzed.

An analysis of cancerous tissue and noncancerous tissue adjacent to the cancerous tissue of patients was carried out for 237 cases which underwent gastrectomy without preoperative chemotherapy in Nagoya University School of Medicine from 2001 to 2012. Tissue samples were rapidly frozen in liquid nitrogen and thereafter stored at −80° C. and analyzed. Table 2 shows the expression of ANOS1, the presence or absence of peritoneal dissemination, and the classification by UICC (the Union for Cancer Control) seventh edition.

Determination of peritoneal dissemination was carried out by a cyto-diagnosis of peritoneal cavity lavage solution, and peritoneal dissemination was determined to be positive or negative. Also, the average value of ANOS1 mRNA expression levels in patient gastric cancer tissues for 237 cases was calculated, expression higher than the average value was deemed to be a high expression of ANOS1, and that lower than the average value was deemed to be low expression of ANOS1. ANOS1 expression was analyzed by quantitative PCR. Quantitative PCR was performed using an ANOS1 primer of the following sequences using an ABI STEPOnePlus Real-Time PCR System, and carrying out analysis by amplification in PCR conditions of 40 cycles of 95° C. 10 seconds and 60° C. 30 seconds, following 95° C. for 10 minutes.

Primer Sequences

ANOS1:
(SEQ ID NO: 7)
Forward AACAATGGTTCCCTGGTTTG
(SEQ ID NO: 8)
Reverse TCACAAAAGCTTTGGCACTG

TABLE 2

| | | ANOS1 High | ANOS1 Low | |
|---|---|---|---|---|
| | | Expression | Expression | |
| | | (Number of Patients) | (Number of Patients) | p value |
| Peritoneal | | 82 | 105 | <0.001 |
| dissemination | | | | |
| positive | | | | |
| Peritoneal | | 36 | 14 | |
| dissemination | | | | |
| negative | | | | |
| UICC Stage | I | 8 | 50 | <0.001 |
| | II | 20 | 20 | |
| | III | 40 | 30 | |
| | IV | 50 | 19 | |

It is apparent from the results in Table 2 that high expression of ANOS1 and peritoneal dissemination positive are correlated, and further, that ANOS1 is highly expressed in patients in more advanced stages.

### Example 12

«Expression Analysis in Cell Lines»

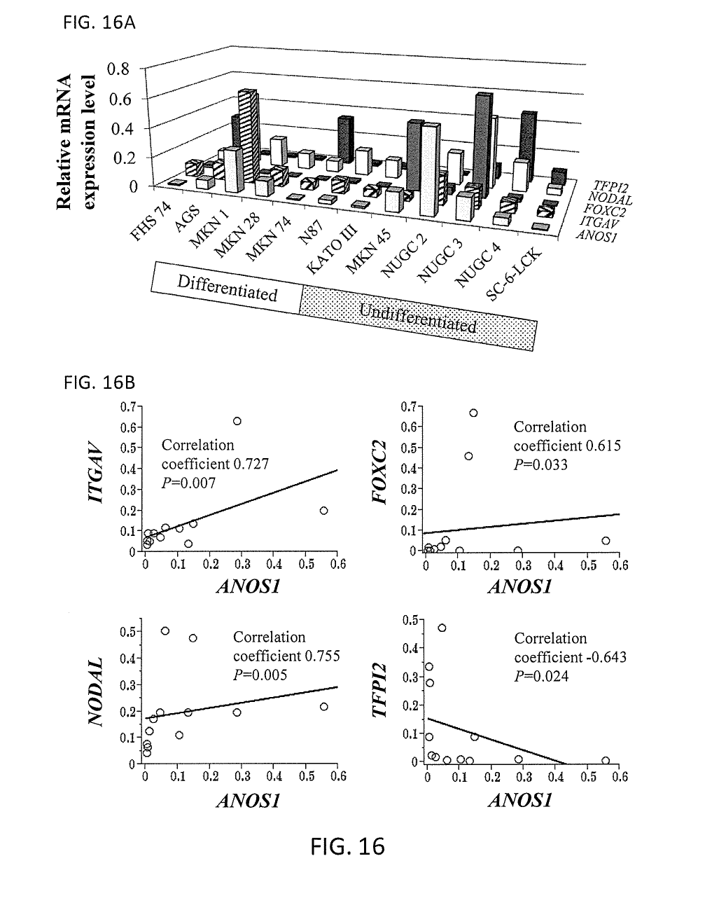

A PCR array analysis was performed on 11 gastric cancer cell lines and non-cancer epithelial cell line FHs74 in the same manner as Example 2. A correlation test was performed between these results and the expression level of the ANOS1 in each cell line. The results are shown in FIG. 16.

Expression of ANOS1 had a significant positive correlation with integrin αV (ITGAV), which is an integrin known to be involved in cell adhesion, EMT-related transcription factor FOXC2, which is critical in metastasis and invasion of cancer cells, and growth differentiation factor NODAL. Also, expression of ANOS1 had a negative correlation with tissue factor pathway inhibitor 2 (TFPI2), for which suppression is often observed in EMT and is known to receive methylation in many gastric cancers. It is suggested from these results that ANOS1 is coordinately expressed with known major cancer-related molecules, and, based on the correlation with these results, possibly promotes gastric cancer peritoneal dissemination.

### Example 13

«Knockdown Analysis Using siRNA»

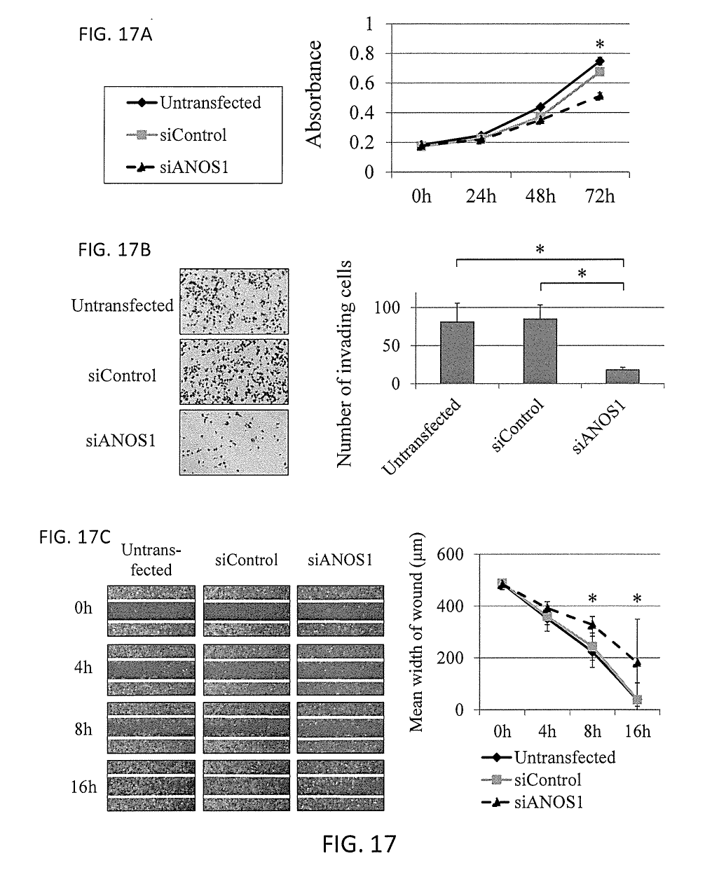

In the same manner as Example 8, an experiment to selectively inhibit of expression (knockdown) of ANOS1 was carried out in vitro using gastric cancer cell lines (MKN1, MKN45), which have high expression of ANOS1, and the proliferation potency, invasion potency, and migratory potency of gastric cancer cells were evaluated. The results are shown in FIGS. 17A-C.

ANOS1 siRNA (Dharmacon Inc.) were transfected to the cells using Accell siRNA transfection methods (Dharmacon, Inc.), the cells were incubated for 72 hours in a serum-free DMEM medium, and the proliferation potency, invasion potency, and migratory potency were evaluated. It was apparent that when expression of ANOS1 is inhibited, there is mild suppression of proliferation potency of gastric cancer cells, and significant suppression of invasion potency and migratory potency.

### Example 14

«Analysis of ANOS1 Protein Expression in Gastric Tissue by Immunohistochemical Staining»

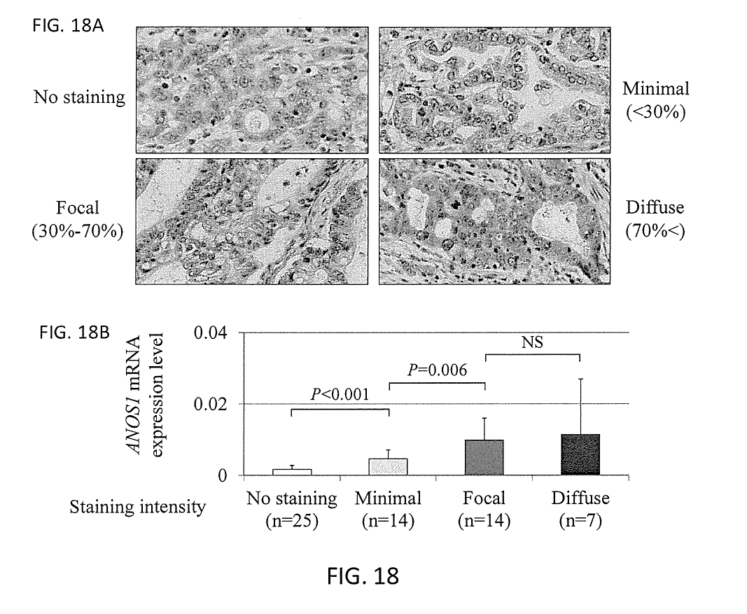

In the same manner as Example 3, ANOS1 protein expression was analyzed using 60 cases of patient tissue. Except for using an anti-ANOS1 antibody (Millipore Corp.), immunohistochemical staining was carried out in the same manner as in Example 3. ANOS1 expression intensities were classified into no staining (No staining, in FIG. 18), minimal (Minimal (<30%), in FIG. 18), focal (Focal (30-70%)), and diffuse (Diffuse (>70%)). FIG. 18A shows a typical staining image. FIG. 18B shows the relationship between ANOS1 mRNA expression levels and the tissue staining images. The ANOS1 mRNA expression level is a value obtained by normalizing the expression level of mRNA with the expression level of GAPDH mRNA.

Regarding the correlation with the expression intensities of ANOS1, tissue specimens were randomly assigned a symbol prior to analysis, and two observers carried out measurement without information about the specimen in order to ensure objectivity. Each observer performed at least two measurements and evaluations for all the specimens to minimize observer differences. ANOS1 mRNA expression levels in tissue and ANOS1 protein expression intensities obtained by immunohistochemical staining were significantly correlated. In the result of analyzing the correlation between ANOS1 mRNA expression levels and protein expression intensities in gastric cancer tissue in the same patient case (FIG. 18B), a significant correlation was observed therebetween, and it was shown that the results of mRNA expression analysis of gastric cancer tissues can be applied to protein expression analysis as well.

### Example 15

«Analysis of Prognosis and ANOS1 mRNA Expression Levels in Tissue»

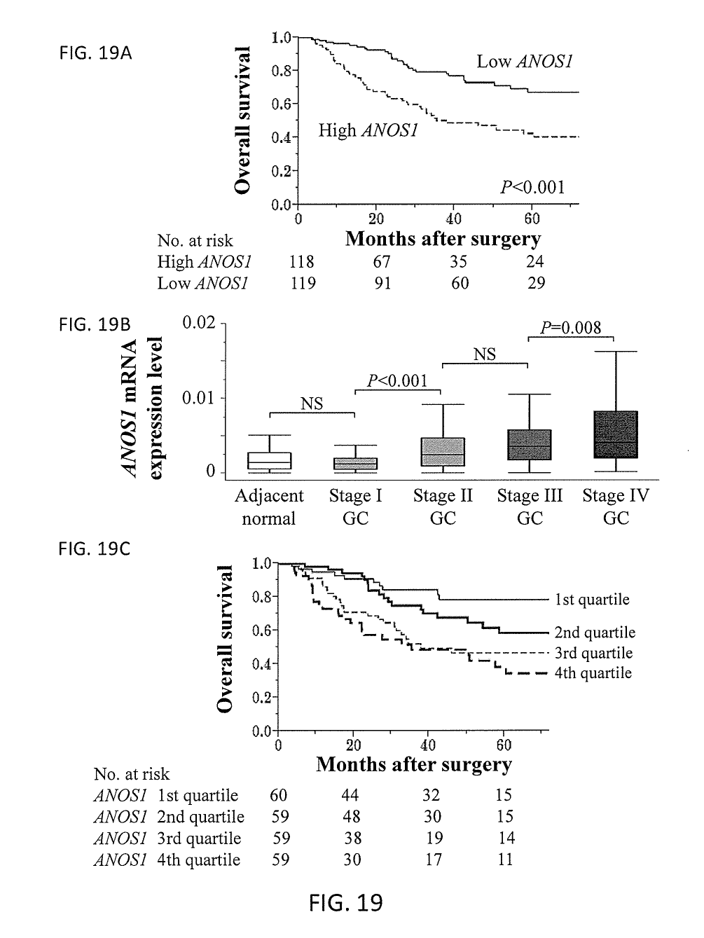

The correlation between prognosis and ANOS1 expression levels in tissue was analyzed. In the same manner as Table 2, patients were classified into ANOS1 high-expression patients and low-expression patients from ANOS1 expression by mRNA in gastric cancer tissue, and the number of survivors after surgery was plotted (FIG. 19A). It was apparent that ANOS1 high-expression patients have significant poor prognoses in comparison with low-expression patients.

A relationship between stage by UICC and ANOS1 mRNA was shown (FIG. 19B). The expression level in ANOS1 in tissue shows an increase in commensurate fashion to the progress of gastric cancer. Furthermore, patients were classified into quartiles of ANOS1 mRNA expression levels in gastric cancer tissue, and the number of survivors after surgery was plotted (FIG. 19C). The plot suggests that survival rates decrease in accordance with higher expression of ANOS1, and therefore that measuring the expression level of ANOS1 in tissue makes it possible to predict a prognosis.

### Example 16

«Analysis of Amount of ANOS1 Protein in Patient Preoperative Serum, and Stage and Prognosis»

ANOS1 in the serum of 60 healthy subjects and 146 gastric cancer patients was analyzed by ELISA. Serum samples that were used were collected within 7 days prior to surgery, and then rapidly frozen to −80° C. and stored. The serum ANOS1 levels were measured using a human ANOS1 ELISA kit (CUSABIO Co.).

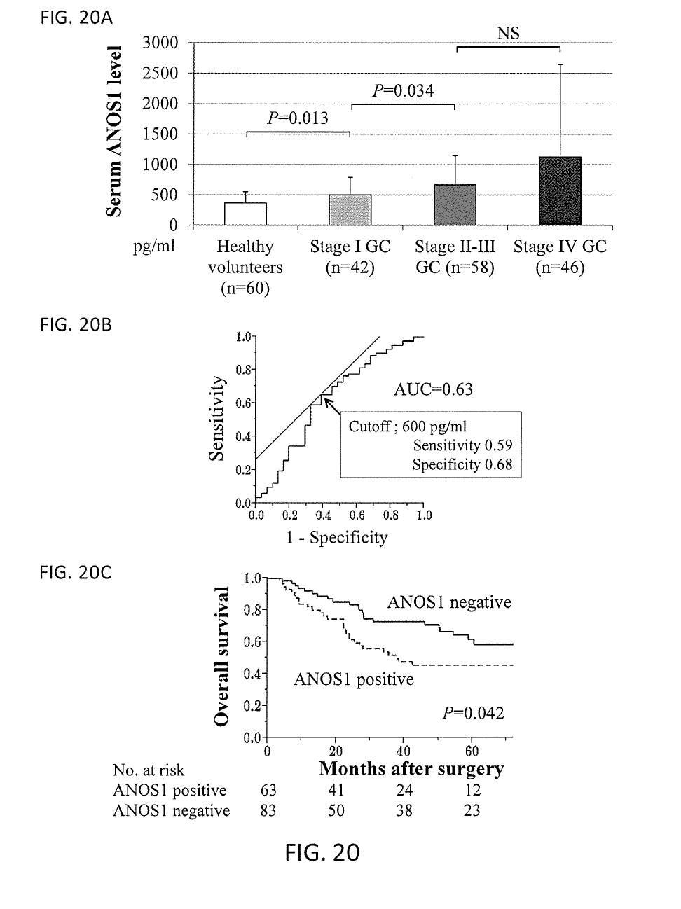

In addition to the serum ANOS1 values being significantly higher values in gastric cancer patients than in healthy subjects, the values were also elevated as stage progresses (FIG. 20A). The cutoff values for ANOS1 values was obtained by ROC curve analysis (FIG. 20B), the gastric cancer patients were divided into two groups by the cutoff values the correlation with prognosis was analyzed. Serum ANOS1 high-value group had significant poor prognoses, as shown in FIG. 20C. In other words, this shows that examining the amount of ANOS1 protein in patient serum makes it possible to predict the stage and prognosis of a patient prior to surgery. Being able to predict the stage and prognosis using the less invasive method of measuring ANOS1 in patient serum has great benefits to the patient as well.

It is apparent from the above results that the presence of peritoneal dissemination and the expression level of ANOS1, and consequently, patient prognosis is significantly correlated. The ANOS1 level can be measured using mRNA in gastric cancer tissue, the amount of protein, and the amount of serum ANOS1 protein. It is therefore possible to predict the possibility of peritoneal dissemination and the prognosis immediately prior to and after surgery, and that therapy can be conducted on the basis of more detailed therapy policies.

### INDUSTRIAL APPLICABILITY

In accordance with the present invention, the expression levels of SYT13, SYT8, and ANOS1 in samples obtained at the time of surgery can be used as a marker for the occurrence of peritoneal dissemination, and it is possible to test postoperative risk. As a result, it is possible to perform the patient therapy on the basis of more detailed therapy policies. Furthermore, it is possible to develop a drug which acts selectively on gastric cancer peritoneal dissemination by screening using the expression levels of SYT13, SYT8, and ANOS1 as indicators. Furthermore, SYT13 and SYT8 siRNA suppressed peritoneal dissemination in peritoneal dissemination model animals, and therefore, it is thought that these siRNAs can directly suppress peritoneal dissemination recurrence.

[Sequence Table]
SYT8_SYT25.txt

1-10. (canceled)

11. A testing method for predicting postgastrectomy peritoneal dissemination, wherein:
a measurement is taken for an expression level of at least one of SYT13, SYT8, and ANOS1 in at least one sample of patient serum, peritoneal cavity lavage solution in a gastrectomy, and gastric tissue, collected from a subject, and
risk of peritoneal dissemination is determined to be high when the expression level of at least one of SYT13, SYT8, and ANOS1 in the sample is higher than a predetermined value.

12. The testing method of claim 11, wherein:
a method for measuring the expression level of SYT13, SYT8, and ANOS1 measures the expression level of SYT13, SYT8, and ANOS1 mRNA and/or protein.

13. The testing method of claim 12, wherein:
the method for measuring the expression level of SYT13, SYT8, and ANOS1 mRNA is carried out by quantitative PCR.

14. A test kit for diagnosing or predicting postgastrectomy peritoneal dissemination, wherein:
the test kit comprises one or more of an anti-SYT13 antibody, an anti-SYT8 antibody, an anti-ANOS1 antibody, and a primer set for quantitative PCR for measuring an expression level of SYT13, SYT8, and ANOS1.

15. A method for screening a molecularly targeted therapeutic agent in order to treat postgastrectomy peritoneal dissemination, wherein:
the method for screening a molecularly targeted therapeutic agent by targeting SYT13, SYT8, ANOS1 comprises screening a substance by using expression, or suppression of function, of at least one of SYT13, SYT8, and ANOS1 as an indicator.

16. The testing method of claim 13, wherein:
a risk of peritoneal dissemination metastasis recurrence is determined to be high when the expression level of mRNA in gastric cancer tissue is equal to or greater than a SYT13 cutoff value of 0.05 or a SYT8 cutoff value of 0.005.

17. The testing method of claim 12, wherein:
a risk of peritoneal dissemination metastasis recurrence in a patient is determined to be high when the expression level of ANOS1 protein serum is a cutoff value 600 pg/mL or greater.


SEQUENCE LISTING (8 sequences)

SEQ ID NO: 1 - DNA, 20 bp, Artificial Sequence
  PCR primer
gcttctctctccggtacgtg

SEQ ID NO: 2 - DNA, 20 bp, Artificial Sequence
  PCR primer
aggaaggtgaaggcctcatt

SEQ ID NO: 3 - DNA, 18 bp, Artificial Sequence
  PCR Primer
acctggagaaggcgaagc

SEQ ID NO: 4 - DNA, 19 bp, Artificial Sequence
  PCR primer
tctgggaacttgaggaggg

SEQ ID NO: 5 - DNA, 19 bp, Artificial Sequence
  PCR preimer
gaaggtgaaggtcggagtc

SEQ ID NO: 6 - DNA, 20 bp, Artificial Sequence
  PCR primer
gaagatggtgatgggatttc

SEQ ID NO: 7 - DNA, 20 bp, Artificial Sequence
  PCR primer
aacaatggttccctggtttg

SEQ ID NO: 8 - DNA, 20 bp, Artificial Sequence
  PCR primer
tcacaaaagctttggcactg